(19) United States
(12) Patent Application Publication
(10) Pub. No.: US 2019/0365320 A1
(43) Pub. Date: Dec. 5, 2019

(54) ORAL DEVICE INCLUDING PH SENSOR

(71) Applicant: Smartooth Korea Co., Ltd., Seoul (KR)

(72) Inventors: Ho Jung Son, Seoul (KR); Ji Young Sim, Seoul (KR)

(21) Appl. No.: 16/395,451

(22) Filed: Apr. 26, 2019

(30) Foreign Application Priority Data
Jun. 1, 2018 (KR) 10-2018-0063669
Aug. 2, 2018 (KR) 10-2018-0090483
Apr. 1, 2019 (KR) 10-2019-0037981

(51) Int. Cl. A61B 5/00; A61B 5/145

(52) U.S. Cl. CPC A61B 5/682; A61B 5/14539; A61B 5/1468; A61B 5/4277; A46B 2200/1066

(57) ABSTRACT

An oral device for checking a time to go to a dentist. The oral device includes a pH sensor configured to measure pH within an oral cavity, a housing on which the pH sensor is mounted, a processor configured to analyze measured pH from the pH sensor, and a wireless communication unit functionally connected to the processor and configured to perform communication with an external device. The oral device is useful for a patient who needs continuous management such as an implant surgery patient by allowing the patient to visually and easily check a time to go to a dentist and an individual oral condition of the patient. Further, by allowing the user to simply check the condition of the oral cavity at home, it is possible to more effectively manage the condition of the teeth and to save medical expenses.

### CROSS REFERENCE TO RELATED APPLICATIONS

This application claims priority to Korean Patent Application No. 10-2018-0063669, filed. Jun. 1, 2018; Korean Patent Application No. 10-2018-0090483, filed Aug. 2, 2018 and Korean Patent Application No. 10-2019-0037981, filed Apr. 1, 2019, the contents of such applications being incorporated by reference herein.

### BACKGROUND OF THE INVENTION

### 1. Field of the Invention

The present disclosure relates to an oral device including a pH sensor. Particularly, the present disclosure relates to an oral device including a pH sensor capable of early diagnosis of inflammation, a decayed tooth, or the like in an oral cavity.

### 2. Description of the Prior Art

When patients go to a dentist and complain of pain, frequently, many of patients are already in the state in which a large part of a tooth needs to be removed or much of the tooth has already been removed. Once a tooth is damaged, the tooth cannot be regrown or restored to its original state.

In particular, periodontal disease is one of the biggest causes of tooth loss, and this also applies to implants. Implant is one of the most difficult procedures to drill a hole in the alveolar bone. When the procedure is wrong, it is difficult to remove the implant attached to the alveolar bone and re-operation is also difficult. Thus, teeth can be preserved longer if they are diagnosed and prevented before severe damage. Particularly, when the inflammation around an implant is detected and treated to minimize the damage of the bone tissue, it is possible to extend the maintenance period of the dental prosthesis.

However, most patients find it, difficult to determine whether or not there is an abnormality in their teeth before they have the pain. In addition, currently, there is no dental equipment or implant that informs a patient in advance of whether or not there is an abnormality in the patient's tooth condition.

### SUMMARY OF THE INVENTION

Accordingly, the inventors have invented an oral device including a pH sensor. The present disclosure aims to provide an oral device that visually shows the oral condition of a patient so that the patient can periodically check his or her tooth condition.

Thus, an aspect of the present disclosure is to provide an oral device capable of checking a time to go to a dentist and diagnosing inflammation and a tooth decay early.

An oral device according to various embodiments of the present disclosure includes a pH sensor configured to measure pH within an oral cavity; a housing on which the pH sensor is mounted; a processor configured to analyze measured pH from the pH sensor; and a wireless communication unit functionally connected to the processor and configured to perform communication with an external device.

In the oral device of the present disclosure, the housing may be any one selected from a group consisting of an implant screw, a toothbrush, an electric toothbrush, and a tip of an oral cleaner.

In the oral device of the present disclosure, the processor may analyze acidity measured by the pH sensor within the oral cavity.

The oral device of the present disclosure may further include a display, and the display may display a measured pH value using visible colors.

In the oral device of the present disclosure, the processor may be configured to: indicate a red color on the display when the measured pH value is less than 5.5; indicate an orange color on the display when the measured pH value is 5.5 to 6.0; indicate a yellow color on the display when the measured pH value is 6.0 to 6.5; and indicate a green color on the display when the measured pH value is 6.5 or more.

In the oral device of the present disclosure, the housing may further include a built-in battery that supplies power to the pH sensor.

In the oral device of the present disclosure, when the housing is the tip of the oral cleaner, the housing may further include an injection nozzle and a water injection unit that injects water at a predetermined pressure.

In the oral device of the present disclosure, when the housing is the tip of the oral cleaner, the oral device may further include a water tank, and the water tank may include a pumping motor configured to drive the water tank by receiving power, and a connection part configured such that water within the water tank is compressed through the pumping motor to be delivered to an injection nozzle within the housing.

In the oral device of the present disclosure, when the housing is the tip of the oral cleaner, the oral device may further include a power supply unit (on/off), a sensor power unit, and a water pressure control unit.

In the oral device of the present disclosure, when the housing is a toothbrush or an electric toothbrush, the pH sensor is mounted on a portion opposite to a toothbrush head.

The oral device provided by the present disclosure is useful for a patient who needs continuous management such as an implant surgery patient by allowing the patient to check a time to go to a dentist and the individual oral condition of the patient. Further, by allowing the user to simply check the condition of the oral cavity at home, it is possible to more effectively manage the condition of the teeth and to save medical expenses.

### BRIEF DESCRIPTION OF THE DRAWINGS

The above and other aspects, features and advantages of the present disclosure will be more apparent from the following detailed description taken in conjunction with the accompanying drawings, in which:

FIG. 1 is a view illustrating an oral device according to an embodiment of the present disclosure;

FIG. 2 illustrates an index showing a pH measurement value;

FIG. 3 is a view illustrating an oral device according to an embodiment of the present disclosure;

FIGS. 4 and 5 are views each illustrating an oral device according to an embodiment of the present disclosure;

FIG. 6 is a block diagram of an oral device according to an embodiment of the present disclosure; and

FIG. 7 is a view illustrating an oral device according to an embodiment of the present disclosure.

### DETAILED DESCRIPTION OF THE EXEMPLARY EMBODIMENTS

An oral device according to various embodiments of the present disclosure includes a pH sensor; a housing on which the pH sensor is mounted; a processor configured to analyze measured pH from the pH sensor; and a wireless communication unit functionally connected to the processor and configured to perform communication with an external device.

The pH sensor may be a small sensor capable of measuring pH in an oral cavity. Through the oral device of the present disclosure, which includes the pH sensor part, a patient is able to check her or his oral condition by herself or himself. Oral diseases, such as dental caries, are caused by hydrogen ionization concentration, saliva buffering ability, saliva viscosity, saliva flow rate, and sensitivity, and these factors are closely related to oral acidity. The average pH of oral acidity is about 7.0 and is neutral. However, when there are gum diseases such as dental caries or periodontitis, oral acidity is lowered. That is, by providing a pH sensor in the oral device in order to use this principle, it is possible to periodically check the presence of inflammation in an oral cavity, -to check a time to go to a dental clinic, and to manage teeth more efficiently.

The pH sensor may be mounted on a housing. At this time, the housing may be any one selected from a group consisting of an implant screw, a toothbrush, an electric toothbrush, and a tip of an oral cleaner.

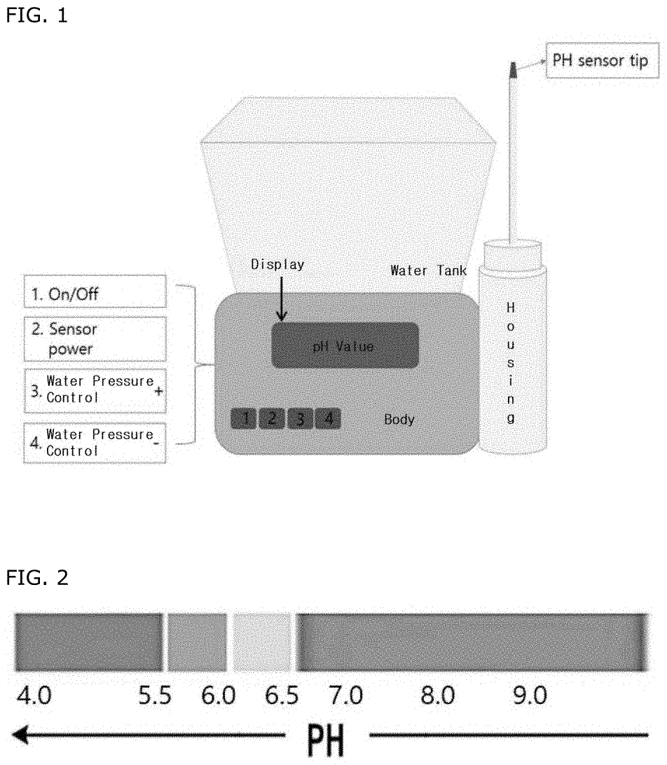

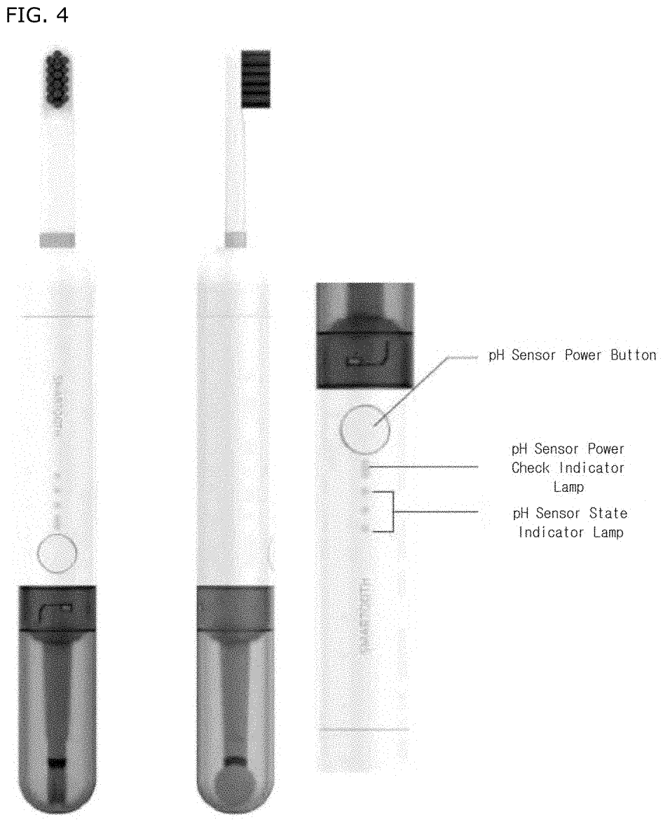

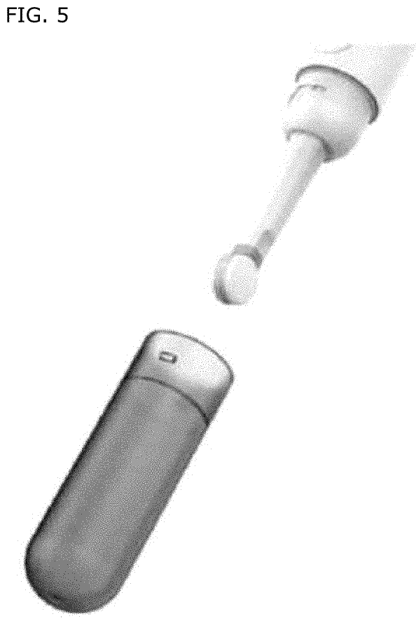

More specifically, as illustrated in FIG. 1, the pH sensor may be mounted on the tip of an oral cleaner. Alternatively, as illustrated in FIG. 3, the pH sensor may be mounted within a toothbrush or an electric toothbrush. Alternatively, as illustrated in FIG. 4, the pH sensor may be mounted within an implant screw.

Meanwhile, the housing may further include a built-in battery that supplies power to the pH sensor.

Meanwhile, the processor may analyze the pH measured from the pH sensor. Specifically, the processor may analyze the acidity in the oral cavity through the pH measured by the pH sensor. In addition, the processor may transmit the analyzed pH data or acidity data to an external device via the wireless communication unit. Such a processor may be a Micro Controller Unit (MCU).

The wireless communication unit is functionally connected to the processor, and may communicate with an external device. The wireless communication unit may transmit the pH data or acidity data analyzed by the processor to the external device.

The wireless communication performed by the wireless communication unit may include at least one of, for example, Wireless Fidelity (Wi-Fi), Bluetooth, Zigbee, Near-Field Communication (NFC), and Radio Frequency (RF).

Meanwhile, the oral device of the present disclosure may further include a display. The display may display measured pH values using visible colors. For example, as illustrated in FIG. 1, the display may be provided on the body of the oral cleaner. Alternatively, as illustrated in FIG. 3, the display may be provided on the handle portion of the toothbrush or the electric toothbrush. Alternatively, as illustrated in FIG. 4, the display may be provided on a separate external apparatus (e.g., a smart phone, a computer, or the like).

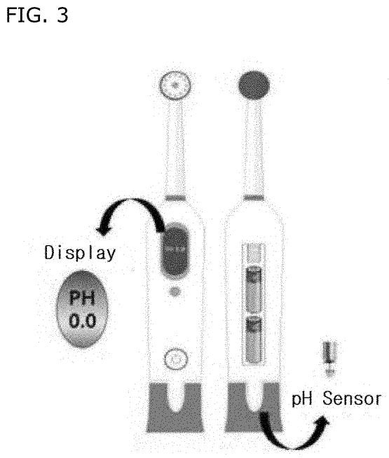

At this time, as illustrated in FIG. 2, the processor may indicate various colors on the display depending on measured pH values. For example, when the measured pH value is less than 5.5, the processor may indicate a red color on the display. When the red color is indicated on the display, a measurer may perform the measurement again after a certain period of time has passed after the toothbrushing or food ingestion (e.g., every 15 minutes), and when the same result is displayed even though the measurement is performed again, the measurer may obtain information that it is necessary to go to a dental clinic.

When the measured pH value is 5.5 to 6.0, the processor may indicate an orange color on the display. At this time, the measurer may obtain information that it is necessary to pay attention to the oral condition.

When the measured pH value is 6.0 to 6.5, the processor may indicate a yellow color on the display. At this time, the measurer may obtain information that it is necessary to continuously monitor the oral condition.

For example, when the measured pH value is 6.5 or more, the processor may indicate a green color on the display. At this time, the measurer may determine that the intraoral state is good.

Since the oral device of the present disclosure indicates a color on the display depending on the measured pH value, the measurer is able to easily check his or her oral condition.

Meanwhile, an oral device according to an embodiment of the present disclosure will be described in more detail with reference to FIG. 1. According to one embodiment of the present disclosure, the oral device may be an oral cleaner.

That is, as described above, the pH sensor may be mounted on the tip of an oral cleaner. At this time, the tip may be a water-pick or an aquapick.

The water-pick or the aquapick is a device used for removing food residue remaining between teeth or between teeth and gingiva by injecting pressurized water onto the teeth.

The pH sensor may be mounted on the tip portion of the housing, which injects the pressurized water into the teeth, and may measure the pH when it is brought into contact with the gingiva.

Meanwhile, the housing may have an injection nozzle mounted therein so that water can be sprayed under pressure, and may include a water injection unit provided at the upper end thereof.

The oral cleaner may further include a water tank. The water tank is connected to the water supply unit as a water supply unit, and pressurizes the water to flow to the housing. The water tank is provided with a pumping motor driven by being supplied with power and includes a connection part so that the water within the water tank can be transmitted to the injection nozzle at a predetermined pressure. The water in the water tank flows into the housing through the driving of the pumping motor and is injected into the water injection unit through an injection nozzle at a predetermined pressure by the pumping motor. By this injection at the predetermined pressure, foreign matter between the teeth and between the teeth and the gums can be removed.

The body of the oral cleaner may further include a power supply unit (on/off), a sensor power unit, and a water pressure control unit.

The power supply unit may be mounted in the form of a button to turn on/off the power, the sensor power unit may be mounted in the form of a button to adjust the power of the pH sensor tip mounted on the tip of the housing, and the water pressure control unit may be control the water pressure and may be configured such that when a button indicated with a “+” mark is pushed, the water pressure increases and when a button indicated with a “−” mark is pushed, the water pressure decreases.

In some cases, the oral cleaner according to the present disclosure may be configured such that the body of the oral cleaner is directly connected to a water tap without including the water tank, so that directly supplied water can be injected. That is, by making the water tap be connected to the body without the water tank, the water can be moved through the injection nozzle within the housing and the water can be injected through the water injection unit at the upper end of the housing.

Referring to FIG. 3, the oral device according to an embodiment of the present disclosure may be a toothbrush or an electric toothbrush.

At this time, the pH sensor may be mounted on a portion of the toothbrush or the electric toothbrush opposite to the toothbrush head. The pH may measure by bringing the pH sensor into contact with the gingiva. In addition, by providing the handle of the toothbrush with a display, a color depending on the pH value may be indicated on the display. The toothbrush head is manufactured as a replacement type so that the sensor can be used continuously.

FIGS. 4 and 5 are views each illustrating an oral device according to an embodiment of the present disclosure. Referring to FIGS. 4 and 5, the oral device according to an embodiment of the present disclosure may be a toothbrush or an electric toothbrush.

Referring to FIG. 4, an oral device according to an embodiment of the present disclosure may include a toothbrush head, a housing, a pH sensor, and a sensor protection cap.

A board having components such as a Low Pass Filter (LPF), an amplifier (Amp), a processor (e.g., MCU), and a wireless communication unit (e.g., Bluetooth) mounted thereon, a battery for providing power, and the like may be accommodated in the housing. In addition, outside the housing, a pH sensor power button, a pH sensor power check LED indicator lamp, and a pH sensor state indicator lamp may be included.

The pH sensor power button may turn on/off the pH sensor. That is, the user may operate the pH sensor or may terminate the operation of the pH sensor by pushing the pH sensor power button.

The pH sensor power check LED indicator lamp may be turned on/off depending on whether or not the pH sensor is operated. That is, when the pH sensor is operated, the pH sensor power check LED indicator lamp is turned on, so that the user can easily determine whether the pH sensor is turned on or off.

The pH sensor state indicator lamp may include at least three indicator lamps indicating different colors. A first indicator lamp may indicate a red color, a second indicator lamp may indicate a yellow color, and a third indicator lamp may indicate a blue color. Each indicator lamp may be turned on depending on the measured pH value. When the measured pH value is 5 or less, the first indicator lamp indicating a red color may be turned on, when the measured pH value is more than 5 and less than 7, the second indicator lamp indicating the yellow color may be turned on, and when the measured pH value is 7 or more, the third indicator lamp indicating the blue color may be turned on. Through this, the user is able to easily check the change of the pH value according to the oral condition.

Meanwhile, referring to FIG. 5, the sensor protection cap is able to protect the pH sensor. The sensor protection cap may have a rounded end. The sensor protection cap may be coupled to the housing. For example, the sensor protection cap may be rotated and coupled to the housing. This may prevent water from penetrating into the housing. The sensor protection cap may be made of at least one of a translucent material, transparent material and opaque material. This makes it possible to visually observe the pH sensor reaching an inner portion of the mouth, thereby giving a clean image.

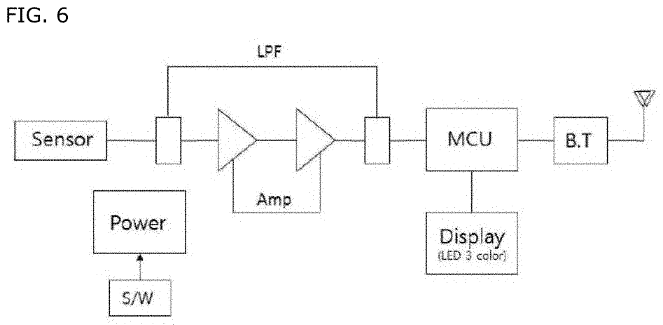

FIG. 6 is a block diagram of an oral device according to an embodiment of the present disclosure.

Referring to FIG. 6, a Low-Pass Filter LPF may filter a signal measured by a pH sensor. The amplifier Amp may amplify the signal filtered through the low pass filter LPF. The amplified signal may be filtered through another low pass filter LPF. The MCU may analyze pH data or acidity data through the filtered signal. The pH data or acidity data analyzed by the MCU may be transmitted to an external device (e.g., a smart phone or a computer) through Bluetooth B.T, which is a wireless communication unit. Meanwhile, the MCU may operate the pH sensor state indicator lamp depending on the analyzed pH data or acidity data. That is, the MCU may operate any one of the first indicator lamp, the second indicator lamp, and the third indicator lamp that indicate different colors depending on the measured pH value.

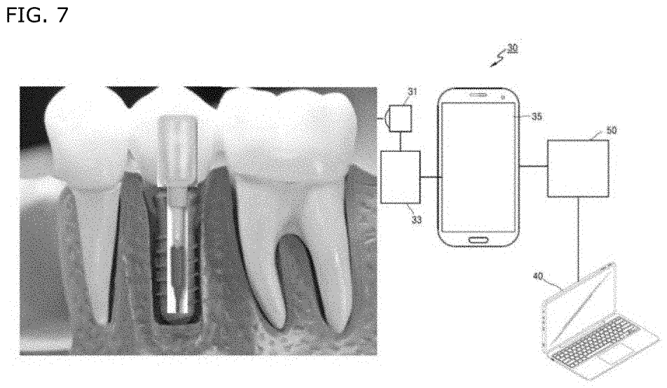

Referring to FIG. 7, the oral device according to an embodiment of the present disclosure may be an implant.

At this time, the pH sensor may be provided within the fixture or screw of the implant. Implants do not have periodontal ligaments unlike teeth. Thus, the implants may stimulate the alveolar bone and may be more likely to cause inflammation. However, since it is difficult to check this state with naked eye, diagnosis should be made using X-ray at a dental clinic. However, with the present disclosure, it is possible to extend the maintenance period of implants, so that the convenience of patients can be improved and medical expenses can be reduced.

The oral device provided by the present disclosure allows a consumer to easily check the state of his/her oral cavity, to make an early diagnosis of inflammation, a decayed tooth, or the like in' the oral cavity, and to individually and conveniently check and determine a time to go to a dentist by causing the intraoral state to be visually displayed.

What is claimed is:

1. An oral device comprising:
a pH sensor configured to measure pH within an oral cavity;
a housing on which the pH sensor is mounted;
a processor configured to analyze the measured pH from the pH sensor; and
a wireless communication unit functionally connected to the processor and configured to perform communication with an external device.

2. The oral device of claim 1, wherein the housing is any one selected from a group consisting of an implant screw, a toothbrush, an electric toothbrush, and a tip of an oral cleaner.

3. The oral device of claim 1, wherein the processor analyzes acidity measured by the pH sensor within the oral cavity.

4. The oral device of claim 1, further comprising a display, wherein the display indicates a measured pH value using a visible color.

5. The oral device of claim 4, wherein the processor is configured to:
indicate a red color on the display when the measured pH value is less than 5;
indicate an yellow color on the display when the measured pH value is 5 to 7; and
indicate a blue color on the display when the measured pH value is 7 or more.

6. The oral device of claim 1, wherein the housing further includes a built-in battery that supplies power to the pH sensor.

7. The oral device of claim 1, wherein, when the housing is a tip of an oral cleaner, the housing further includes an injection nozzle and an injection unit that inject water at a predetermined pressure.

8. The oral device of claim 1, wherein, when the housing is a tip of an oral cleaner, the oral device further includes a water tank, and
wherein the water tank includes:
a pumping motor configured to receive power to drive the water tank; and
a connection part configured such that water within the water tank is compressed through the pumping motor to be delivered to an injection nozzle within the housing.

9. The oral device of claim 1, wherein, when the housing is a tip of an oral cleaner,
the oral device further comprises a power supply unit (on/off), a sensor power unit, and a water pressure control unit.

10. The oral device of claim 1, wherein, when the housing is a toothbrush or an electric toothbrush,
the pH sensor is mounted on a portion opposite to a toothbrush head.
